(19) United States
(12) Patent Application Publication
(10) Pub. No.: US 2026/0345298 A1
(43) Pub. Date: Nov. 5, 2026

(54) DIGITAL STOCK WROUGHT WIRE CLASP, REST SEAT, AND ORTHODONTIC LIBRARY FOR 3D PRINTING AND MILLING DENTAL APPLIANCES

(71) Applicant: Regents of the University of Minnesota, Minneapolis, MN (US)

(72) Inventors: John MADDEN, Minneapolis, MN (US); Paul OLIN, Minneapolis, MN (US)

(21) Appl. No.: 19/125,570

(22) PCT Filed: Oct. 31, 2023
(86) PCT No.: PCT/US2023/036404

§ 371 Date: Apr. 29, 2025

Related U.S. Application Data
(60) Provisional application No. 63/420,989, filed on Oct. 31, 2022.

(51) Int. Cl. A61C 13/00; A61C 13/01; A61C 13/08; A61C 13/225; A61C 13/267

(52) U.S. Cl. CPC A61C 13/0004; A61C 13/0019; A61C 13/01; A61C 13/082; A61C 13/2255; A61C 13/267

(57) ABSTRACT

A method of fabricating a removable dental appliance includes the steps of: (a) acquiring 3D digital image data of a patient's mouth and dentition; (b) creating a 3D digital model of a removable dental appliance to be fabricated wherein the 3D digital model incorporates a model clasp and/or model seat selected from a digital library of virtual clasps and/or seats, each of the virtual clasps or seats corresponding to an associated physical clasp or seat; (c) fabricating a portion of the removable dental appliance from the 3D digital model, the portion of the removable dental appliance including an attachment section; (d) selecting a physical clasp or seat that corresponds to the model clasp or seat; and (e) inserting a tail section of a selected physical clasp and/or seat into mounting structure in the attachment section, the tail section being dimensioned to matingly engage the mounting structure.

### CROSS-REFERENCES TO RELATED APPLICATIONS

The present application is based on, claims priority to, and incorporates herein by reference in its entirety for all purposes, U.S. Patent Application No. 63/420,989, filed Oct. 31, 2022.

### STATEMENT REGARDING FEDERALLY SPONSORED RESEARCH

Not Applicable.

### FIELD OF THE INVENTION

This invention introduces a simplified workflow that expedites the clinical and laboratory steps of a manufacturing process of a removable dental appliance and ultimately renders a product that can be produced directly from intraoral scans and three dimensional (3D) printing.

### BACKGROUND

Various removable dental appliances are known. For example, a partial denture is a prosthesis that replaces one or more, but not all of the natural teeth and supporting structures. It is supported by the teeth and/or the mucosa. It may be fixed (i.e., a bridge) or removable. A removable partial denture (RPD) is a partial denture that can be removed and replaced in the mouth by the patient. RPDs are generally indicated for partially edentulous patients who cannot have fixed prostheses due to, for example, health conditions, cost or aesthetics considerations, and the extent and position of the edentulous span. RPDs are supported and retained by the remaining natural teeth (referred to as “abutment teeth”), tissue and/or implants.

Historically, the partial denture has been prototyped and manufactured in a long and drawn out process requiring many long dental appointments and a large number of steps where error can be introduced in the clinic or laboratory. Two of the large sources of error are using stone (plaster) models and the warping of acrylic during heat processing.

The first dental appointment traditionally begins with a set of alginate impressions of the patients remaining teeth. These impressions are then made into stone (plaster) models. The models are then analyzed using a process called “surveying”. This is used to determine the ideal path of insertion for the proposed partial denture. At this time, the rest seats for the frame are also planned and incorporated into the design.

After the design is planned, the patient has their second appointment. At this appointment, the rest seats and guide planes are prepared in the patient's teeth using a dental drill. New impressions are taken using alginate, and stone (plaster) models are poured again.

Both the models with the proposed design and the final models from the second appointment are sent to the laboratory for a chrome cobalt alloy metal frame to be fabricated. To fabricate the metal frame, a wax pattern of the frame is made on the stone model using pre-made wax pieces specifically grid work, clasps, and major connectors. The wax patterns are formed to fit the dental arch and match the proposed design.

The frame is then removed from the cast and invested. It is typically cast out of cobalt chrome alloy. After it is cast, the frame is divested and laboriously finished, polished, and fit back to the stone model. It is then returned to the dentist to be fit in the patient's mouth.

After the 3rd dental appointment (when the frame is fit in the patient's mouth), it is returned to the dental lab for “wax-rims” to be attached to the edentulous spaces on the frame. The frame with the wax-rim is then returned to the dentist for the jaw relations appointment.

At the 4th appointment, the wax-rims are adjusted to determine the correct vertical dimension of occlusion (VDO) and record the correct jaw relations or bite. All the components are then sent back to the laboratory so the prototype tooth arrangement can be made with pre-made carded denture teeth (relatively expensive).

At the 5th dental appointment, the dentist and the patient approve the proposed tooth arrangement and make any adjustments. If necessary, a new bite is taken, and the case is remounted and adjustments are made in the laboratory.

After the prototype tooth arrangement is finalized, the model, the frame, and the tooth arrangement are flasked in stone (plaster) making a two-part mold. This mold is used to pack polymethyl methacrylate (PMMA acrylic) into the correct shape around the frame and secure the teeth.

After curing for 8 hours at 165 °F, the acrylic is solid and is divested from the mold. It is then trimmed, finished, and polished to be delivered to the patient.

At the 6th appointment, the final removable partial denture is fitted to the patient's mouth making any last minute adjustments. After wearing this for several days, there may be 1-2 follow-up appointments for adjusting sore spots.

It is estimated that some 12 million people in the United States are fully edentulous. On their path to losing all their teeth, they most likely have worn one or several removable partial dentures, leading to millions of these dental appliances being made in the United States each year.

The fabrication of removable partial dentures as described above often involves a complicated traditional workflow accompanied by ample room for human error. Furthermore, design-induced errors present themselves in abundance as the rudiments of traditional design become less and less familiar to the general dentist and technician. This is largely due to the fact that the basics of the partial denture are complex and the hours and repetition required to master these concepts are not found in today's dental education.

In addition to faulty design cues, many errors come from the traditional way RPD production uses heat-activated polymethyl methacrylate denture-base resin pressed to stone. These stone models are duplicated several times throughout the course of the fabrication introducing error and discrepancy with each duplication. The crippling tendency of acrylic shrinkage and contraction during thermal polymerization and the error of expansion and contraction of stone amplified over several layers of duplication yields frustrating results. However frustrating, the field of removable prosthodontics has been employing this wax-loss pattern and flasking technique for over a century. A step forward to replacing stone casts and pressed packed acrylics with digital design and 3D printing is long overdue.

In addition to detrimental effects of duplicating stone models and acrylic shrinkage, many tedious patient-dentist visits are inevitable to acquire physical records of oral anatomy to ensure proper fitment on the day of delivery. There are usually 3-5 appointments with the introduction of large error or failure at each. Each appointment also is separated by many calendar days if not weeks and along with time for shipping.

Various solutions to the foregoing problems associated with preparing a removable partial denture are disclosed in PCT Patent Application Publication No. WO 2021/262849 A2. However, even further technological advances are desirable in methods for fabricating a removable dental appliance, such as a removable partial denture.

### SUMMARY OF THE DISCLOSURE

In one aspect, the present disclosure provides a method of fabricating a removable dental appliance that engages an abutment tooth. The method includes the steps of: (a) acquiring 3D digital image data of a patient's mouth and dentition; (b) creating a 3D digital model of a removable dental appliance to be fabricated wherein the 3D digital model incorporates a model clasp selected from a digital library of virtual clasps, each of the virtual clasps corresponding to an associated physical clasp of a plurality of physical clasps structured to accommodate functional movement of the removable dental appliance to be fabricated with respect to the abutment tooth when the removable dental appliance to be fabricated is placed in the patient's mouth; (c) fabricating a portion of the removable dental appliance from the 3D digital model, the portion of the removable dental appliance including an attachment section; (d) selecting one physical clasp of the plurality of physical clasps that corresponds to the model clasp; and (e) inserting a tail section of a selected physical clasp into mounting structure in the attachment section of the removable dental appliance, the tail section being dimensioned to matingly engage the mounting structure in the attachment section to assemble the selected physical clasp to the attachment section of the removable dental appliance.

In one embodiment of the method, each of the plurality of physical clasps comprises at least one section comprising wrought wire. In one embodiment of the method, each of the plurality of physical clasps comprises a ball clasp. In one embodiment of the method, each ball clasp comprises a horizontal member attached to a tail component and a vertical member that terminates in a ball.

In one embodiment of the method, the horizontal member of at least two of the plurality of physical clasps has a different length. In one embodiment of the method, the vertical member of at least two of the plurality of physical clasps has a different length. In one embodiment of the method, the tail component of at least one of the plurality of physical clasps has a looped configuration or a bent configuration. In one embodiment of the method, the tail component of at least one of the plurality of physical clasps is stamped. In one embodiment of the method, the tail component of at least one of the plurality of physical clasps includes one or more cutout regions within a perimeter of the tail component. In one embodiment of the method, the tail component of at least one of the plurality of physical clasps has an abraded surface. In one embodiment of the method, the tail component of at least one of the plurality of physical clasps has a serrated edge.

In one embodiment of the method, each of the plurality of physical clasps comprises a C-clasp. In one embodiment of the method, each C-clasp comprises a ridgelap segment attached to a tail component and a curved portion. In one embodiment of the method, the curved portion at least one of the plurality of physical clasps terminates in a ball. In one embodiment of the method, the ridgelap segment of at least two of the plurality of physical clasps has a different length. In one embodiment of the method, the curved portion of at least two of the plurality of physical clasps has a different length. In one embodiment of the method, the tail component of at least one of the plurality of physical clasps has a looped configuration or a bent configuration. In one embodiment of the method, the tail component of at least one of the plurality of physical clasps is stamped. In one embodiment of the method, the tail component of at least one of the plurality of physical clasps includes one or more cutout regions within a perimeter of the tail component. In one embodiment of the method, the tail component of at least one of the plurality of physical clasps has an abraded surface. In one embodiment of the method, the tail component of at least one of the plurality of physical clasps has a serrated edge.

In one embodiment of the method, each of the plurality of physical clasps comprises a reciprocal arm and a retentive arm wherein at least one of the reciprocal arm and the retentive arm comprises wrought wire.

In one embodiment of the method, step (e) comprises curing a resin in the mounting structure in the attachment section to assemble the selected physical clasp to the attachment section of the removable dental appliance.

In one embodiment of the method, step (c) comprises fabricating the portion of the removable dental appliance using 3D printing.

In one embodiment of the method, step (c) comprises fabricating the portion of the removable dental appliance using a milling procedure.

In one embodiment of the method, step (e) comprises inserting the tail section of the selected physical clasp into mounting structure in the attachment section of the removable dental appliance during the milling procedure.

In one embodiment of the method, each of the plurality of physical clasps is selected from the group consisting of ball clasps, C-clasps, wing clasps, I-bar clasps, Y-bar clasps, combination clasps, RPI clasps, RPA clasps, rest seats, orthodontic clasps, Adams clasps, Hawley retainers, and palatal expanders.

In one embodiment of the method, the removable dental appliance is a removable partial denture comprising (i) a frame sized and shaped to conform to a mouth inner surface; and (ii) a tooth segment assembled to the frame, and the attachment section is a base section of the frame.

In one embodiment of the method, the mounting structure in the attachment section comprises a cut out.

In one embodiment of the method, the 3D digital model incorporates a model seat selected from a digital library of virtual seats, each of the virtual seats corresponding to an associated physical seat of a plurality of physical seats.

In one embodiment of the method, the method further comprises: (f) selecting one physical seat of the plurality of physical seats that corresponds to the model seat; and (g) inserting a tail section of a selected physical seat into mounting structure in the attachment section of the removable dental appliance, the tail section of the selected physical seat being dimensioned to matingly engage the mounting structure in the attachment section to assemble the selected physical seat to the attachment section of the removable dental appliance.

In one embodiment of the method, step (c) comprises fabricating the portion of the removable dental appliance using 3D color printing.

In one embodiment of the method, the removable dental appliance is a removable partial denture comprising (i) a frame sized and shaped to conform to a mouth inner surface; and (ii) a tooth segment assembled to the frame, the attachment section is a base section of the frame, and step (e) comprises inserting the tail section of the selected physical clasp into mounting structure in a buccal flange of the base section of the frame.

In one embodiment of the method, the removable dental appliance is a removable partial denture comprising (i) a frame sized and shaped to conform to a mouth inner surface; and (ii) a tooth segment assembled to the frame, the attachment section is a base section of the frame, and step (e) comprises inserting the tail section of the selected physical clasp into mounting structure in a lingual flange of the base section of the frame.

In another aspect, the present disclosure provides a method of fabricating a removable dental appliance that engages an abutment tooth. The method includes the steps of: (a) acquiring 3D digital image data of a patient's mouth and dentition; (b) creating a 3D digital model of a removable dental appliance to be fabricated wherein the 3D digital model incorporates a model seat selected from a digital library of virtual seats, each of the virtual seats corresponding to an associated physical seat of a plurality of physical seats; (c) fabricating a portion of the removable dental appliance from the 3D digital model, the portion of the removable dental appliance including an attachment section; (d) selecting one physical seat of the plurality of physical seats that corresponds to the model seat; and (e) inserting a tail section of a selected physical seat into mounting structure in the attachment section of the removable dental appliance, the tail section being dimensioned to matingly engage the mounting structure in the attachment section to assemble the selected physical seat to the attachment section of the removable dental appliance. The method can further comprise: (f) creating a preparation in the abutment tooth using a dental bur wherein the preparation has an interior surface for matingly engaging an outer surface of the selected physical seat wherein the dental bur has a cutting edge geometry that matches the outer surface of the selected physical seat.

In one embodiment of the method, the method further comprises: (f) creating a preparation in the abutment tooth wherein the preparation has an interior surface for matingly engaging an outer surface of the selected physical seat. In one embodiment of the method, step (f) comprises creating the preparation in the abutment tooth using a dental bur. In one embodiment of the method, the dental bur has a cutting edge geometry that matches the outer surface of the selected physical seat. In one embodiment of the method, the dental bur has a depth stop.

In one embodiment of the method, the method further comprises: (f) providing a plurality of dental burs, one or more of the dental burs having a cutting edge geometry that matches an outer surface of one or more of the plurality of physical seats; and (g) creating a preparation in the abutment tooth using a least one of the dental burs, wherein the preparation has an interior surface for matingly engaging an outer surface of the selected physical seat.

In one embodiment of the method, the tail section of at least one of the plurality of physical seats has a looped configuration or a bent configuration. In one embodiment of the method, the tail section of at least one of the plurality of physical seats is stamped. In one embodiment of the method, the tail section of at least one of the plurality of physical seats includes one or more cutout regions within a perimeter of the tail component. In one embodiment of the method, the tail section of at least one of the plurality of physical seats has an abraded surface. In one embodiment of the method, the tail section of at least one of the plurality of physical seats has a serrated edge.

In one embodiment of the method, step (e) comprises curing a resin in the mounting structure in the attachment section to assemble the selected physical seat to the attachment section of the removable dental appliance.

In one embodiment of the method, step (c) comprises fabricating the portion of the removable dental appliance using 3D printing.

In one embodiment of the method, step (c) comprises fabricating the portion of the removable dental appliance using a milling procedure.

In one embodiment of the method, step (e) comprises inserting the tail section of the selected physical seat into mounting structure in the attachment section of the removable dental appliance during the milling procedure. In one embodiment of the method, the mounting structure in the attachment section comprises a cut out.

In one embodiment of the method, the removable dental appliance is a removable partial denture comprising (i) a frame sized and shaped to conform to a mouth inner surface; and (ii) a tooth segment assembled to the frame, and the attachment section is a base section of the frame.

In one embodiment of the method, the 3D digital model incorporates a model clasp selected from a digital library of virtual clasps, each of the virtual clasps corresponding to an associated physical clasp of a plurality of physical clasps.

In one embodiment of the method, the method further comprises: (f) selecting one physical clasp of the plurality of physical clasps that corresponds to the model clasp; and (g) inserting a tail section of a selected physical clasp into mounting structure in the attachment section of the removable dental appliance, the tail section of the selected physical clasp being dimensioned to matingly engage the mounting structure in the attachment section to assemble the selected physical clasp to the attachment section of the removable dental appliance.

In one embodiment of the method, step (c) comprises fabricating the portion of the removable dental appliance using 3D color printing.

In yet another aspect, the present disclosure provides a method of fabricating a removable partial denture that engages an abutment tooth. The method comprises: (a) acquiring 3D digital image data of a patient's mouth and dentition; (b) creating a 3D digital model of a removable partial denture to be fabricated wherein the 3D digital model incorporates a model clasp structured to accommodate functional movement of the removable partial denture to be fabricated with respect to the abutment tooth when the removable partial denture to be fabricated is placed in the patient's mouth; (c) fabricating a portion of the removable partial denture from the 3D digital model, the portion of the removable partial denture including a tooth attachment section and at least one artificial tooth; (d) fabricating an additional artificial tooth having an attached physical clasp that corresponds to the model clasp; and (e) assembling the additional artificial tooth in the tooth attachment section of the removable partial denture.

In one embodiment of the method, the physical clasp comprises at least one section comprising wrought wire.

In one embodiment of the method, the physical clasp comprises a ball clasp.

In one embodiment of the method, the physical clasp comprises a C-clasp.

In one embodiment of the method, the physical clasp comprises a reciprocal arm and a retentive arm wherein at least one of the reciprocal arm and the retentive arm comprises wrought wire.

In one embodiment of the method, step (e) comprises curing a resin in the tooth attachment section to assemble the physical clasp in the tooth attachment section of the removable partial denture.

In one embodiment of the method, step (c) comprises fabricating the portion of the removable partial denture using 3D printing.

In one embodiment of the method, step (c) comprises fabricating the portion of the removable partial denture using color 3D printing.

In one embodiment of the method, step (c) comprises fabricating the portion of the removable partial denture using milling.

In one embodiment of the method, step (d) comprises fabricating the additional artificial tooth having the attached physical clasp using 3D printing.

In one embodiment of the method, step (d) comprises fabricating the additional artificial tooth having the attached physical clasp using color 3D printing.

In one embodiment of the method, step (d) comprises fabricating the additional artificial tooth having the attached physical clasp using milling.

The foregoing and other aspects and advantages of the invention will appear from the following description. In the description, reference is made to the accompanying drawings which form a part hereof, and in which there is shown by way of illustration example embodiments of the invention. Such embodiments do not necessarily represent the full scope of the invention, however, and reference is made therefore to the claims, drawings, and description herein for interpreting the scope of the invention.

### BRIEF DESCRIPTION OF DRAWINGS

FIG. 1A is a top view of a prior art orthodontic retainer having ball clasps.

FIG. 1B is a side view of a removable partial denture having a C-clasp.

FIG. 1C is a top view of a prior art orthodontic retainer having C-clasps.

FIG. 2 is a side view of a ball clasp suitable for use in a method according to the present disclosure.

FIG. 3A is a partial side view of another ball clasp suitable for use in a method according to the present disclosure.

FIG. 3B is a partial side view of another ball clasp suitable for use in a method according to the present disclosure.

FIG. 3C is a partial side view of another ball clasp suitable for use in a method according to the present disclosure.

FIG. 3D is a partial side view of another ball clasp suitable for use in a method according to the present disclosure.

FIG. 3E is a side view of another ball clasp suitable for use in a method according to the present disclosure.

FIG. 3F is a side view of another ball clasp suitable for use in a method according to the present disclosure.

FIG. 3G is a side view of another ball clasp suitable for use in a method according to the present disclosure.

FIG. 3H is a side view of another ball clasp suitable for use in a method according to the present disclosure.

FIG. 4A is a side view of another ball clasp suitable for use in a method according to the present disclosure.

FIG. 4B is a side view of another ball clasp suitable for use in a method according to the present disclosure.

FIG. 5 is a side view of a C-clasp suitable for use in a method according to the present disclosure.

FIG. 6 is a distal view of the C-clasp of FIG. 5.

FIG. 7 is a side view of another C-clasp suitable for use in a method according to the present disclosure.

FIG. 8A is a side view of another C-clasp suitable for use in a method according to the present disclosure.

FIG. 8B is a side view of another C-clasp suitable for use in a method according to the present disclosure.

FIG. 8C is a side view of another C-clasp suitable for use in a method according to the present disclosure.

FIG. 9 is a side view of another C-clasp suitable for use in a method according to the present disclosure.

FIG. 9A is a partial side view of a tail component of another C-clasp suitable for use in a method according to the present disclosure.

FIG. 9B is a partial side view of a tail component of another C-clasp suitable for use in a method according to the present disclosure.

FIG. 9C is a partial side view of a tail component of another C-clasp suitable for use in a method according to the present disclosure.

FIG. 9D is a partial side view of a tail component of another C-clasp suitable for use in a method according to the present disclosure.

FIG. 9E is a partial side view of a tail component of another C-clasp suitable for use in a method according to the present disclosure.

FIG. 10A is a partial side view of a tail component of another C-clasp suitable for use in a method according to the present disclosure.

FIG. 10B is a partial side view of a tail component of another C-clasp suitable for use in a method according to the present disclosure.

FIG. 11 is a side view of another ball clasp suitable for use in a method according to the present disclosure.

FIG. 12 is a flowchart showing a process of the present disclosure for fabricating a removable partial denture.

FIG. 13A is a perspective view of a premade rest seat suitable for use in a method according to the present disclosure.

FIG. 13B is a mesial view of the premade rest seat of FIG. 13A.

FIG. 14A is a top view of a preparation in an abutment tooth.

FIG. 14B is a distal view of a preparation in an abutment tooth.

FIG. 14C is a top view of a dental bur suitable for use in a method according to the present disclosure.

FIG. 15A is a side view of a dental bur suitable for use in a method according to the present disclosure.

FIG. 15B is another side view of the dental bur of FIG. 15A.

FIG. 15C is a top view of the dental bur of FIG. 15A.

FIG. 16A is a lingual view of a chevron rest suitable for use in a method according to the present disclosure.

FIG. 16B is an occlusal view of an embrasure rest suitable for use in a method according to the present disclosure.

FIG. 16C is a side view of a dental bur suitable for use in a method according to the present disclosure.

FIG. 17A is a side view of a dental long bur suitable to be used horizontally to create a trough between two molars.

FIG. 17B is a side view of a dental round end bur to be used at half its depth to create two dimples for rest seats attached to the embrasure rest.

FIG. 18A is a side view of a dental bur suitable for preparing a distal and/or mesial rest seat.

FIG. 18B is a side view of a dental bur suitable for preparing a distal and/or mesial rest seat.

FIG. 19 is a top view of a removable partial denture according to the present disclosure.

FIG. 20 is a top view of another removable partial denture according to the present disclosure.

FIG. 21 is a top view of another removable partial denture according to the present disclosure.

Like reference numerals will be used to refer to like parts from Figure to Figure in the following description of the drawings.

The invention will be better understood, and features, aspects and advantages other than those set forth above will become apparent when consideration is given to the following detailed description thereof. Such detailed description makes reference to the drawings.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

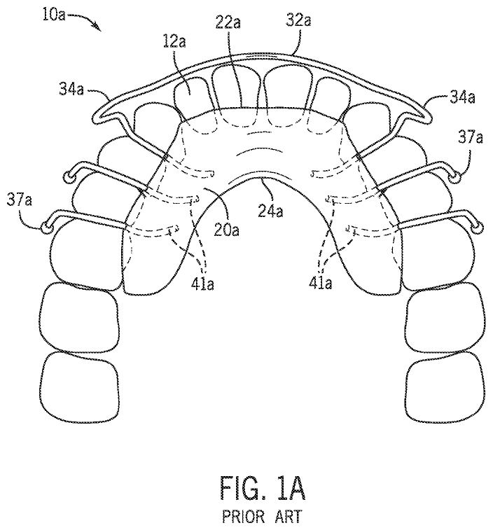

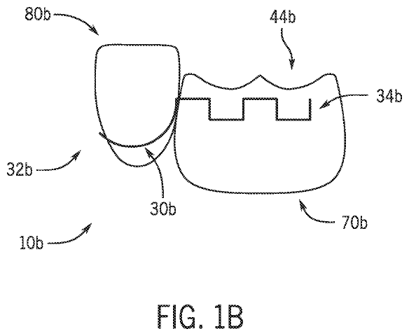

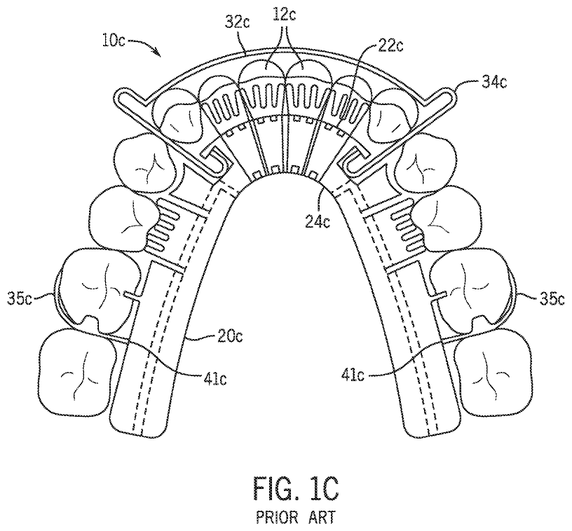

In order to provide context for the invention, examples of removable dental appliances are shown in FIGS. 1A, 1B, and 1C. FIG. 1A shows an orthodontic retainer 10a having ball clasps 37a. Each ball clasp 37a is attached at its tail component 41a to a palatal portion 20a having a rear edge 24a and extend forwardly between teeth 12a such as cuspids. A first ball clasp 37a will generally extend along a first side of a tooth 12a such as a cuspid and a second ball clasp 37a will generally extend along a second side of the tooth 12a for ensuring proper retention and alignment. The orthodontic retainer system 10a has a labial wire 32a which is attached at both ends to the palatal portion 20a of the orthodontic retainer 10a. The labial wire 32a will generally be welded to the palatal portion 20a in a manner which retains the teeth 12a of a patient between the labial wire 32a and the front edge 22a of the palatal portion 20a. The labial wire 32a also includes at least one arch wire 34a for gripping by the patient.

Turning to FIG. 1B, a removable partial denture 10b includes a wrought wire C-clasp 30b embedded into a base section 70b of the frame underneath the tooth segment. The C-clasp 30b has an end 32b engaging the abutment tooth 80b. The C-clasp 30b is bent with a tail component 34b that has a specific design running parallel (horizontally) to the bottom of the tooth segment 44b that allows it only to be adhered and related in one direction. This method can be applied to an all acrylic removable partial denture. Further details can be found in PCT Patent Application Publication No. WO 2021/262849 A2, which is incorporated by reference as if set forth in its entirety herein for all purposes.

Referring now to FIG. 1C, there is shown an orthodontic retainer 10c having C-clasps 35c. Each C-clasp 35c is attached at its tail component 41c to a palatal portion 20c which has a front edge 22c and a rear edge 24c. Each C-clasp 35c will generally extend along a side of a tooth for ensuring proper retention and alignment.

The orthodontic retainer system 10c has a labial wire 32c which is attached at both ends to the palatal portion 20c of the orthodontic retainer 10c. The labial wire 32c will generally be welded to the palatal portion 20c in a manner which retains the teeth 12c of a patient between the labial wire 32c and the front edge 22c of the palatal portion 20c. The labial wire 32c also includes at least one arch wire 34c for gripping by the patient.

A common critical issue in creating digital removable appliances is fitting clasps and attachments into the removable dental appliance when using software to design and print or mill. This disclosure is directed to making a library of pre-made pre-sized clasps to be used in the software for the designs of removable appliances.

One version of the method of the invention includes three components:
1. A physical library of pre-sized clasps (e.g., “C-Clasps” and “Ball Clasps”) that include at least one section comprising wrought wire. The libraries include different sizes and tail configurations that fit the patient in a “best fit matching” fashion. The physical clasps can be provided in a package having separate compartments for each size and tail configuration of clasp.
2. A digital library of virtual clasps compatible with a dental design software product wherein the digital virtual clasps each correspond to one of the physical pre-bent/pre-sized clasps in the physical library. This digital library also includes appropriate offsets, relational elements, eyelets, as well as best fit matching ability to the spline. A manufacturers DME file or attachment library might be most appropriate but is not limited to this.
3. A unique assembly method that does not require a printed model. Most often, self-cure acrylics of other resins cure to the printed model ruining the appliance and model. A model-less assembly method is necessary to reduce waste, labor, and damage to the model and appliance. This unique model-less assembly method uses the relational elements in the software to ensure proper placement of the clasp to be luted/cured in the proper position as well as adjusted chair-side by the dentist.

### The Physical Library

An example physical library includes, without limitation:
1. Ball Clasps—Nightguards, Partials and Retainers
2. “C” Clasps—Partials
3. Flexible “Wing Clasp” Stock Library—Any Flexible Pre-Made Clasps
4. Pre-Made “Chrome Style”—Chrome or Titanium I-Bars, Y-Bars, C-Clasps or Clasp and Rest Assemblies
5. Premade rest seats
6. Orthodontic clasps, expanders, retainers etc.: including but not limited to Adams clasps, Hawley retainers, and palatal expanders.

### Ball Clasp Library Method

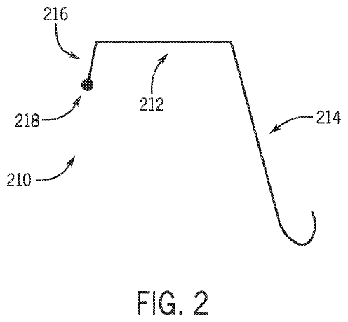

The size of a ball clasp can be broken into three components for a size library for the digital virtual clasps which each correspond to one of the physical pre-bent/pre-sized clasps in the physical library. See FIG. 2 which shows a ball clasp 210 having a horizontal member 212 attached to a tail (retentive/relational) component 214, and a vertical member 216 that terminates in a ball 218.

To make a simple physical library of pre-made ball clasps, there needs to be different dimensions of both the vertical and horizontal members. A measurement of the patient via scan of physical model can help to determine these size measurements for “best fit”. An example library can include a small, small-medium, medium-large, and large values for each member and appropriate tail component.

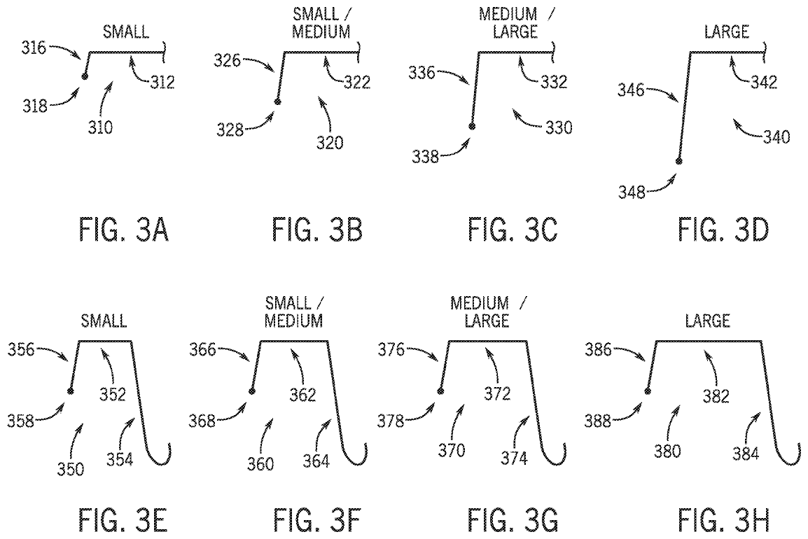

FIG. 3A is a partial side view of another ball clasp 310 having a horizontal member 312 attached to a tail (retentive/relational) component (not shown), and a small vertical member 316 that terminates in a ball 318.

FIG. 3B is a partial side view of another ball clasp 320 having a horizontal member 322 attached to a tail (retentive/relational) component (not shown), and a small-medium vertical member 326 that terminates in a ball 328.

FIG. 3C is a partial side view of another ball clasp 330 having a horizontal member 332 attached to a tail (retentive/relational) component (not shown), and a medium-large vertical member 336 that terminates in a ball 338.

FIG. 3D is a partial side view of another ball clasp 340 having a horizontal member 342 attached to a tail (retentive/relational) component (not shown), and a large vertical member 346 that terminates in a ball 348.

FIG. 3E is a side view of another ball clasp 350 having a small horizontal member 352 attached to a tail (retentive/relational) component 354, and a vertical member 356 that terminates in a ball 358.

FIG. 3F is a side view of another ball clasp 360 having a small-medium horizontal member 362 attached to a tail (retentive/relational) component 364, and a vertical member 366 that terminates in a ball 368.

FIG. 3G is a side view of another ball clasp 370 having a medium-large horizontal member 372 attached to a tail (retentive/relational) component 374, and a vertical member 376 that terminates in a ball 378.

FIG. 3H is a side view of another ball clasp 380 having a large horizontal member 382 attached to a tail (retentive/relational) component 384, and a vertical member 386 that terminates in a ball 388.

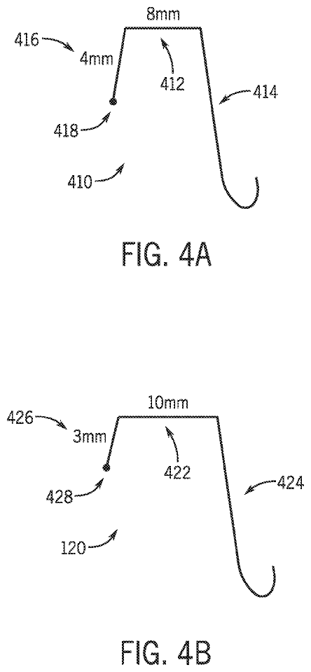

Another method is to have a millimeter value for each member vertically and horizontally giving two numerical values that when added identify the correct pre-made clasp in the library. See the non-limiting examples in FIGS. 4A and 4B. In FIG. 4A, there is shown a clasp 410 having a tail component 414, a ball 418 with a vertical member 416 of 4 millimeters (mm) and a horizontal member 412 of 8 mm which would lead to a 4-8 clasp in the library (or a 48 clasp). In FIG. 4B, there is shown a clasp 420 having a tail component 424, a ball 428 with a vertical member 426 of 3 millimeters (mm) and a horizontal member 422 of 10 mm which would be a 3-10 clasp (or 310 clasp).

### C-Clasp Library Method

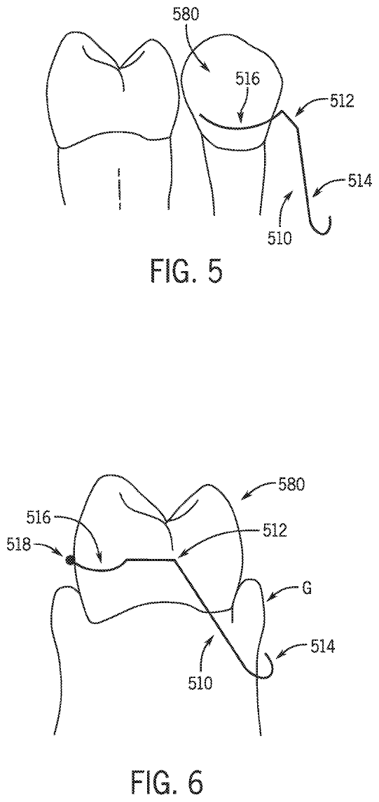

A pre-made C-Clasp can be dissected into its constituent components as well. Again, there are three (or more) possible parts. In FIGS. 5 and 6, a C-Clasp 510 includes a “C” curved portion 516 connected to a ridgelap segment 512 of height of contour of distal contact of the tooth 580, which is connected to a tail retentive/relational component 514. An optional ball 518 may be at the end of the “C” portion 516. The “C” portion 516 engages the tooth 580 adjacent gingiva G.

These three segments as shown in FIGS. 5 and 6 can then be broken down into sizes to form a stock, pre-made wire library for C-Clasps for the digital virtual clasps which each correspond to one of the physical pre-bent/pre-sized clasps in the physical library. The segments (previously referred to as members) that have different sizes are the “C” portion, the ridgelap segment, and the tail retentive/relational component. For this example library, the “C” portion and the “ridgelap” segment can determine which selection is best fit matching.

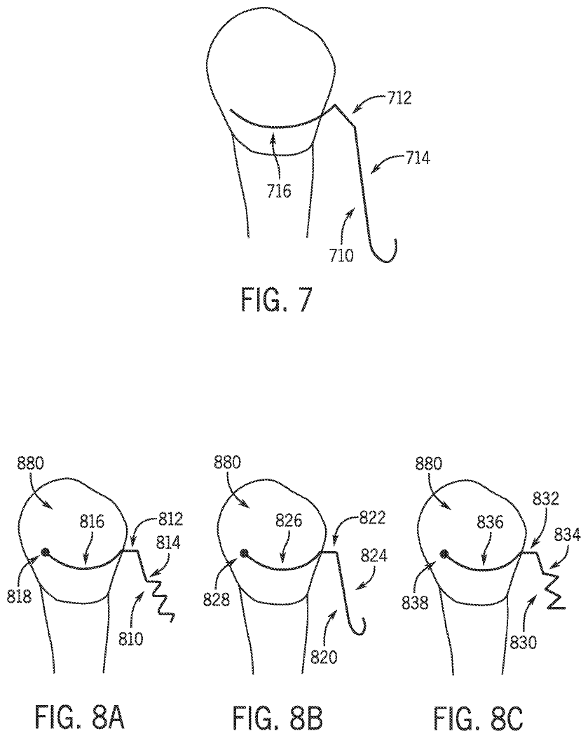

Looking at FIG. 7, a possible way to organize this library may be by measuring in millimeters the length of the “C” portion 716 on a curve for the first value and the length of the ridgelap segment 712 for the second value. This creates a library number as in our previous ball clasp example. In the example C-Clasp 710 of FIG. 7, the “C” portion 716 curve is 9 millimeters (mm) and the ridgelap segment 712 is 3 mm such that the corresponding library value would be 9-3 or 93 or C93.

### Tail End Geometry

The tail end component “geometry/design” of either the ball clasp or the C-clasps must be designed with the explicit intent for it to be assembled with the other printed parts without extraneous printed relational models (study model/assembly only model). The tail end component is important to the model-less assembly.

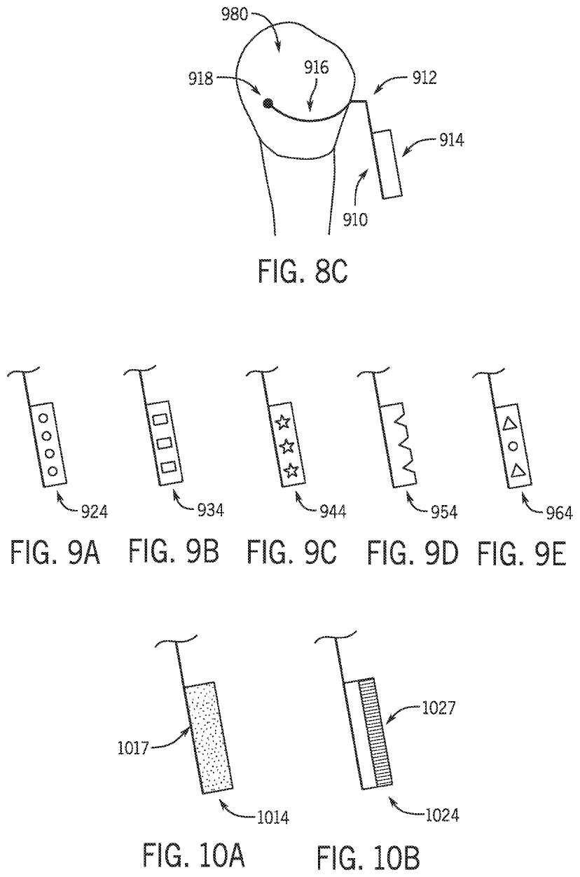

Adding precise relational ability can really be achieved by several different designs; loop or design bent, stamped and cut, or flat and serrated. See FIGS. 8A-8C for examples of tail end component variations.

In FIG. 8A, a C-Clasp 810 includes a “C” portion 816 connected to a ridgelap segment 812, which is connected to a tail retentive/relational component 814. An optional ball 818 may be at the end of the “C” portion 816. The “C” portion 816 engages a tooth 880.

In FIG. 8B, a C-Clasp 820 includes a “C” portion 826 connected to a ridgelap segment 822, which is connected to a tail retentive/relational component 824. An optional ball 828 may be at the end of the “C” portion 826. The “C” portion 826 engages a tooth 880.

In FIG. 8C, a C-Clasp 830 includes a “C” portion 836 connected to a ridgelap segment 832, which is connected to a tail retentive/relational component 834. An optional ball 838 may be at the end of the “C” portion 836. The “C” portion 836 engages a tooth 880.

While these retentive loop patterns might allow for placement without a model, in most cases they are hand set and do not precisely correspond to a designed cut out in the printed base of a removable dental appliance. Having a pre-made library allows for an exact match to be printed in the base of a removable dental appliance. The clasp can either be custom bent by machine or pre-made.

A stamped tail end of the clasp may result in easier placement in the printed base/frame of the removable dental appliance and have the advantage of a thinner, lower profile shape. This leaves more vertical space for a tooth segment of a removable partial denture by being the most common limiting factor.

A stamped tail section may look like (but is not limited to) the configurations in the examples of FIGS. 9 to 9E. In FIG. 9, a C-Clasp 910 includes a “C” portion 916 connected to a ridgelap segment 912, which is connected to a tail retentive/relational component 914. An optional ball 918 may be at the end of the “C” portion 916. The “C” portion 916 engages a tooth 980. The stamped tail end may also be cut to a precise length or have shapes cut into it to aid in retention/relation in a cut out of an attachment section of a removable dental appliance. See the tail retentive/relational components 924, 934, 944, 954, 964 in FIGS. 9A to 9E, respectively.

One further possible variation for the tail component is to add micro retention either through air abrasion of the surface (see the abraded surface 1017 of tail component 1014 in FIG. 10A) or edge serration (see the edge serrations 1027 of tail component 1024 in FIG. 10B).

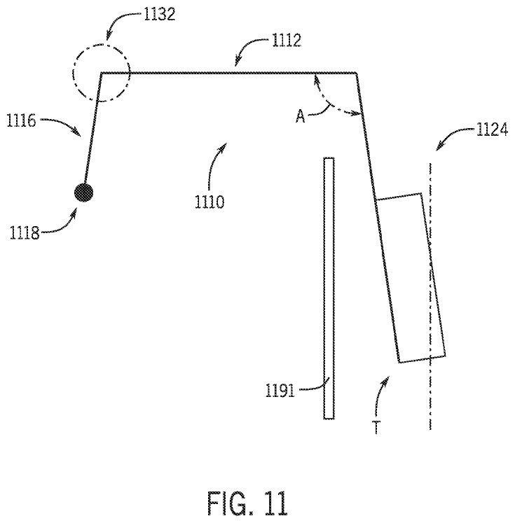

Looking at FIG. 11, the angle A of the tail component may not fit sufficiently to be parallel with the palate requiring it to be adjusted. Note the thickness T of the appliance parallel to the palatal wall 1191. An eyelet design aids in this adjustment. In FIG. 11, a ball clasp 1110 has a horizontal member 1112 attached to a tail (retentive/relational) component 1124, and a vertical member 1116 that terminates in a ball 1118. The clasp wire can be bent so it may only be inserted and embedded into the base section of the removable partial denture (or palatal portion of the orthodontic retainer) in one way. This may require an eyelet 1132 where the clasp wire exits the base section to aid in orientation of the wire. This may include any shape of common ‘tail’ or ‘loop’ on the end of the clasp wire that is embedded in the base section. The shape of the tail may include, for example, a common ‘s’ shaped tail, a square tail, a rectangular tail, a hairpin tail, a loop tail, or a triangular tail. The angle A may be adjusted before luting into place.

The method of the invention is particularly advantageous when used with a clasp that is structured to accommodate functional movement of the removable dental appliance with respect to the abutment tooth when the removable dental appliance is placed in the patient's mouth. For example, in a distal-extension removable partial denture, functional forces applied to the denture base can create an axis of rotation around the distal abutment tooth and a clasp (such as a cast circumferential clasp or an Akers clasp) can act as a wrench and torque the tooth. Specifically, a clasp that comprises a reciprocal arm and a retentive arm may act together to torque a clasped abutment tooth. In the method of the invention, to minimize the torquing effect of the reciprocal arm and the retentive arm, at least one of the reciprocal arm and the retentive arm comprises wrought wire which provides a stress relieving effect resulting in greater flexibility and less ability to torque. Certain clasps may be beneficial in their stress relieving effect, such as an RPI (mesial rest, distal proximal plate and I-bar) clasp, an RPA (mesial rest, distal proximal plate and wrought wire clasp) clasp, and a combination clasp (which uses a cast reciprocal arm and a retentive arm fabricated from a wrought wire). Whenever a cast pure metal or alloy is permanently deformed in any manner it is considered a wrought metal. Because of plastic deformation, the microstructure of an alloy is altered and the alloy exhibits properties that are different from those it had in the as-cast state. The most significant changes in a wrought metal are its increased proportional limit. The proportional limit is the highest stress (the ratio of the applied force to the cross-sectional area of the material it is applied to) at which the stress-strain curve is a straight line. The greater the proportional limit of the wire, the higher the elastic deflection that the wire can deliver. A benefit of wrought wires is that they have a greater elastic deflection than their cast counterparts. Example wrought metals are stainless steel, cobalt-chromium-nickel, and nickel-titanium.

### Denture Rest Seat and Corresponding Bur

To allow for a pre-made rest seat for a partial denture, the dentist must precisely prepare (or carve) the rest seat shape in the abutment tooth (the tooth to receive the rest seat and clasp assembly). This could be done by hand or by “eye” but the desired intimate fit may not be achieved. The clasp library and the rest library work together as they occupy the same space. This is accomplished by having the tail segment be identical or mate and fit in the base with each other or on top of each other.

To ensure the correct shape is achieved, a specialized bur must be used. This bur, or set of burs, is specially designed to correspond with the premade rest-seat library. Different sized burs and rest seats are required to fit all situations and permutations. Different applications of certain burs (i.e., off angle, contra-angle, etc.) must be applied clinically to achieve each configuration of premade rest and clasp assembly.

The width and depth of the preparation in the tooth must be predictable and accurate for a premade rest seat to work. Therefore, the bur must have a predetermined shape and diameter. In addition, a vertical stop or other depth stop may be desirable to achieve the correct fit of the premade rest seat.

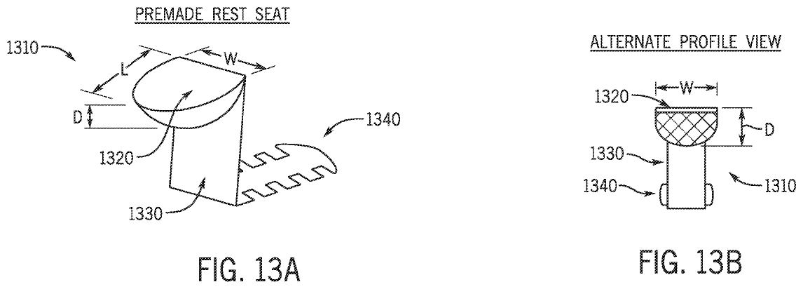

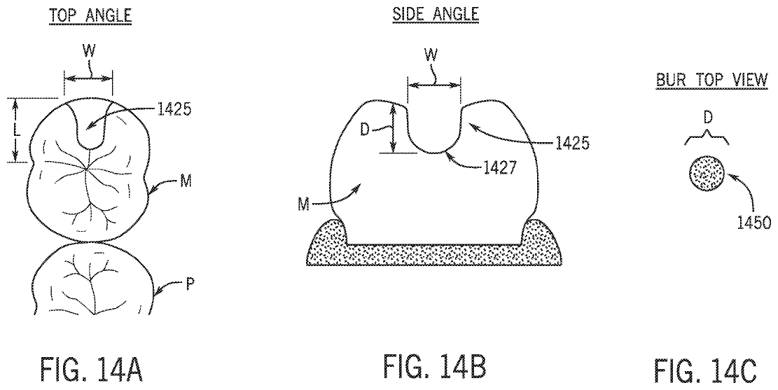

As shown in FIGS. 13A, 13B, 14A, 14B, and 14C, the bur must match the premade rest seat in several dimensions for the resulting tooth preparation to match. The length L, width W, and depth D all must be repeatable and consistent using the burs. Referring to FIGS. 13A and 13B, a premade rest seat 1310 includes a horizontal member 1320 of length L, width W, and depth D, a vertical member 1330 serving as a guide plane, and a retentive tail 1340 having a shape that complementarily mates with a portion of the base section of a removable partial denture. Turning to FIGS. 14A and 14B, there is shown a preparation 1425 in an abutment molar M adjacent premolar P. The preparation 1425 has length L, width W, and depth D, and of has a bottom surface curve 1427 matching a dental bur 1450 in FIG. 14C.

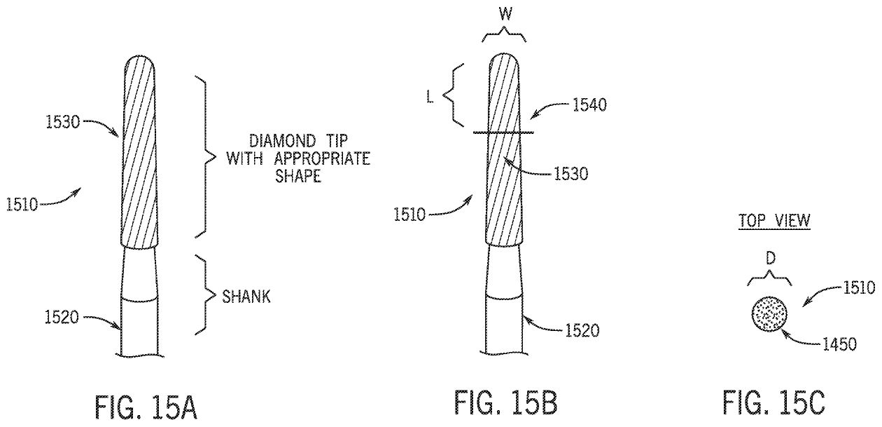

Looking at FIGS. 15A, 15B, and 15C, the premade rest seat, the rest seat in the digital library, and the bur sets must all match exactly. FIGS. 15A, 15B, and 15C show a bur 1510 having a shank 1520, a diamond cutting tip 1530, and a laser line or ring stop 1540. The bur 1510 has length L, width W, and depth D.

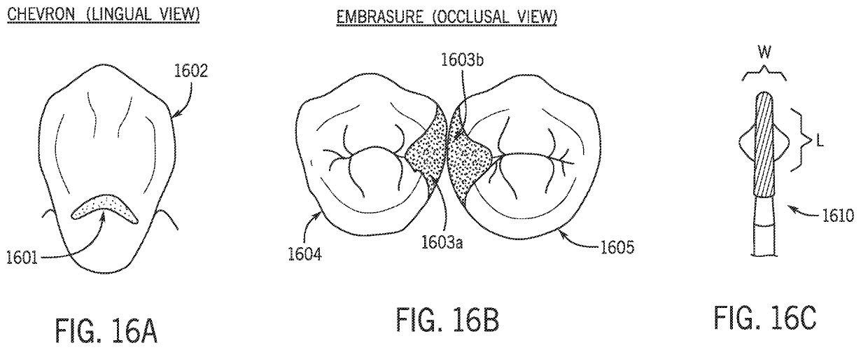

Multiple burs may be required to make additional styles of rest seat including a chevron rest 1601 in tooth 1602 (see FIG. 16A) and embrasure rests 1603a, 1603b in teeth 1604, 1605 (see FIG. 16B). FIG. 16C shows a bur 1610 having a shank 1620, and a cutting tip 1630 for preparing the rest seats. The bur 1610 has cutting length L, cutting width W, and cutting depth D.

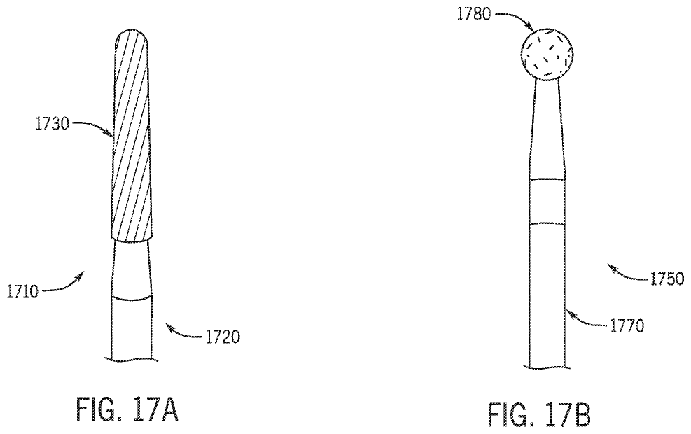

The technique for creating an embrasure rest, such as embrasure rests 1603a, 1603b in teeth 1604, 1605 in FIG. 16B, may contain two types of dental burs: one as a long cylinder bur 1710 having a shank 1720, and a cutting tip 1730 to be used horizontally to create the trough between two molars (see FIG. 17A) and the other as a round-end bur 1750 having a shank 1770, and a round cutting tip 1780 to be used . at half its depth to create two dimples for rest seats attached to the embrasure rest (see FIG. 17B).

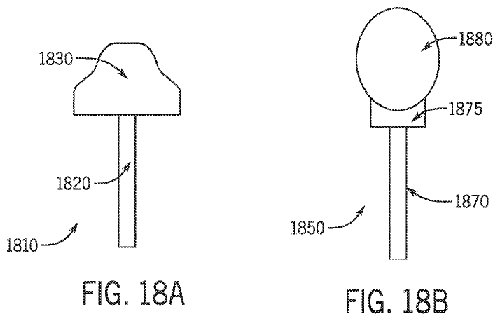

FIG. 18A is a side view of a dental bur 1810 suitable for use in the invention for preparing a distal and/or mesial rest seat. The bur 1810 has a shank 1820, and a wide mouth shaped cutting tip 1830. FIG. 18B is a side view of another dental bur 1850 suitable for use in the invention for preparing a distal and/or mesial rest seat. The bur 1850 has a shank 1870, an intermediate section 1875, and an egg-shaped cutting tip 1880.

### Assembly, Workflow, and Parts Useful for Full Color Printing

Three dimensional (3D) printing in full color is possible in dentistry due to biocompatible poly-jet and multi-jet resins entering the market. A dental design workflow using a clasp library according to the invention can be applied specifically to this printing method. Clasp parts according to the invention can be adapted to fit in a monolithic denture by making a clasp that is embedded in the buccal flange or the lingual flange of the partial denture. The clasp parts for embedment will have a different shape than other clasp parts according to the invention, utilizing a tail that is embedded in the buccal flange or lingual flange (tissue side).

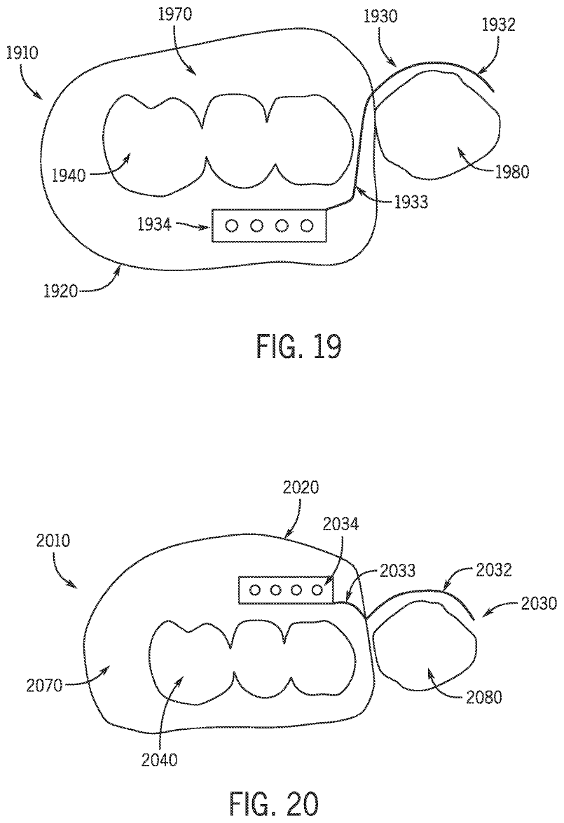

Looking at FIG. 19, a removable partial denture 1910 includes a wrought wire C-clasp 1930 embedded into a lingual flange 1920 of a base section 1970 underneath a tooth segment 1940 of the removable partial denture 1910. The C-clasp 1930 has a “C” portion 1932 engaging the abutment tooth 1980. The “C” portion 1932 is connected to an intermediate portion 1933, which is connected to a tail component 1934 that has a specific design running parallel (horizontally) to the bottom of the tooth segment 1940 that allows it to be embedded in the base section 1970 and related in one direction. This method can be applied to an all acrylic removable partial denture. Further details can be found in PCT Patent Application Publication No.

### WO 2021/262849 A2.

Turning to FIG. 20, a removable partial denture 2010 includes a wrought wire C-clasp 2030 embedded into a buccal flange 2020 of a base section 2070 underneath a tooth segment 2040 of the removable partial denture 2010. The C-clasp 2030 has a “C” portion 2032 engaging the abutment tooth 2080. The “C” portion 2032 is connected to an intermediate portion 2033, which is connected to a tail component 2034 that has a specific design running parallel (horizontally) to the bottom of the tooth segment 2040 that allows it to be embedded in the base section 2070 and related in one direction. This method can be applied to an all acrylic removable partial denture. Further details can be found in PCT Patent Application Publication No. WO 2021/262849 A2.

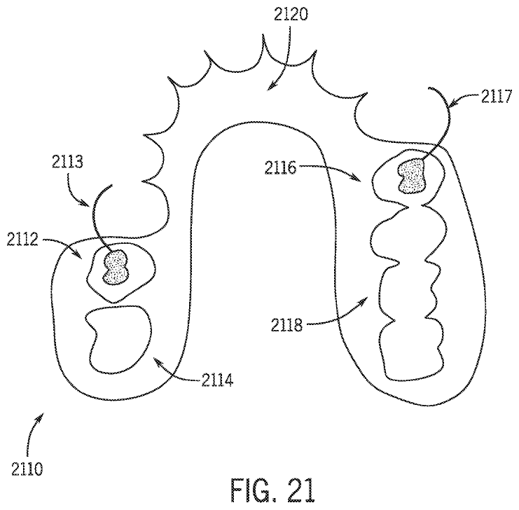

Yet another possibility us to have the clasp parts fit into the bottoms of the denture teeth, but to alter the software design workflow to only have the denture teeth with the clasps in them printed separately. The remaining denture teeth are printed with the denture base in a “monolithic” fashion. Referring to FIG. 21, there is shown a removable partial denture 2110 including artificial teeth 2112, 2114, 2116, and an artificial tooth segment 2118. The artificial tooth 2112 has a wrought wire clasp 2113 secured to or printed with the artificial tooth 2112. The artificial tooth 2112 and the attached wrought wire clasp 2113 can be printed as a separate part to be assembled into a base section 2120 of the removable partial denture 2110. The artificial tooth 2116 has a wrought wire clasp 2117 secured to or printed with the artificial tooth 2116. The artificial tooth 2116 and the attached wrought wire clasp 2117 can be printed as a separate part to be assembled into the base section 2120 of the removable partial denture 2110. The artificial tooth 2114 and the artificial tooth segment 2118 can be printed in the base section 2120 of the removable partial denture 2110 in a “monolithic” fashion before assembly of the artificial teeth 2112, 2116 to base section 2120 of the removable partial denture 2110.

### Parts Library Used in Oversized Milling

The clasp parts library can be used in design for the manufacturing methods of “oversized milling” and “mill and fill” (puck filling/puck remanufacturing). In these embodiments, the parts would be used and inserted and fixed in the milling pucks during the milling procedure, using an interrupted Computer-Aided Manufacturing.

In one oversized milling process, the tooth segment and the base section of the removable partial denture are partially milled with the tooth segment necks and the tooth segment sockets milled to precision. The tooth segment and the base section and the wrought wire clasp are then bonded together, and the removable partial denture is put back in the mill for a final milling.

### EXAMPLE

The following Example is provided in order to demonstrate and further illustrate certain embodiments and aspects of the present disclosure and is not to be construed as limiting the scope of the disclosure.

Digital removable partial dentures began with our attempts to make RPD (removable partial denture) frameworks on site rather than outsourcing them to commercial laboratories. This is essentially the center of the movement in dentistry toward 3D printing in general. Dentists and clinics enjoy the idea of cutting out the middleman and the promise of 3D printing allows in office or on-site production.

Largely, this is a matter of cost, convenience, and a general push toward same day dentistry.

Normally, a cobalt chrome RPD framework requires ten working days or two calendar weeks to get back from the dental laboratory. It is very common to need to rush this timeline for scheduling or emergency purposes and this causes additional fees. Thus, we were searching for a solution that produces RPD frames on-site saving time and money.

The idea that is paramount in digital dentistry is the ability to scan and design the prosthetics from intraoral scan data only. This eliminates completely the need for stone models. In order to achieve this lofty goal with the removable partial denture, the next logical step was to somehow eliminate the need for setting the teeth in wax and conventional acrylic processing eliminating the stone model required to process acrylic to. Therefore, we started to incorporate the tooth colored acetal teeth into the acetal frame design resulting in a one-piece RPD design. This is where many other people developing methods are still at, with a single piece acrylic or acetal design.

At this point, it was believed that there was a market demand for a digital RPD solution because the acetal frames were working very well and catching on quickly. However, it was also obvious that the acetal frame method still required a stone model and traditional steps. The single piece acetal method improved on the clinical and laboratory workflows, but it had its own problems with aesthetics and rigidity of the major connector for large edentulous spaces. Therefore, our focus changed to yet another solution.

The next solution produces a three part RPD using a frame (metal, acetal, PEEK, etc.) and an (acrylic) tooth segment, and a pre-made clasp (such as one of the example embodiments described above). A clasp is typically soldered to a cobalt chrome alloy frame or embedded in acrylic. This applies to RPDs that include a metal frame and all acrylic RPDs. Typically, a stone model is required to shape the wire. One purpose of our RPD concept is to use no stone model.

The clasp assembly problem is solved by the steps of: (a) acquiring 3D digital image data of a patient's mouth and dentition; (b) creating a 3D digital model of an RPD to be fabricated wherein the 3D digital model incorporates a model clasp selected from a digital library of virtual clasps, each of the virtual clasps corresponding to an associated physical clasp of a plurality of physical clasps structured to accommodate functional movement of the RPD to be fabricated with respect to the abutment tooth when the RPD to be fabricated is placed in the patient's mouth; (c) fabricating the frame and tooth segment(s) of the RPD from the 3D digital model, wherein the frame of the RPD includes an attachment section); (d) selecting one physical clasp of the plurality of physical clasps that corresponds to the model clasp; and (e) inserting a tail section of a selected physical clasp into mounting structure (such as a cutout) in the attachment section of the frame of the RPD, wherein the tail section is dimensioned to matingly engage the mounting structure (such as a cutout) in the attachment section to assemble the selected physical clasp to the attachment section of the frame of the RPD.

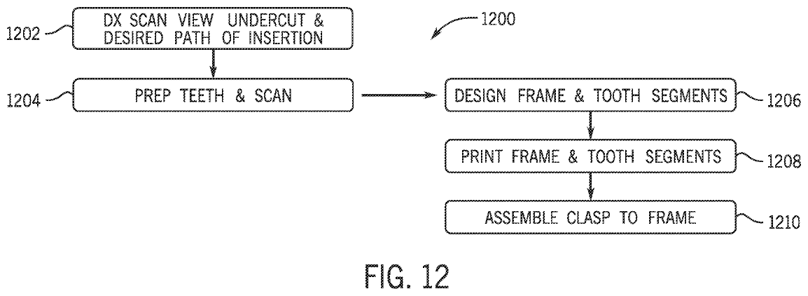

FIG. 12 is a flowchart showing an example process 1200 of the present disclosure for fabricating a removable partial denture (RPD). At step 1202, an intraoral scanner diagnosis (DX) is used to view the undercut and the desired path of insertion.

At step 1204, the teeth are prepped, and a scan is taken with the intraoral scanner which can be a 3D measurement system that is able to capture information on the shape and size of dental arches and to reproduce 3D models of the teeth and soft tissues of the oral cavity, thus allowing complete digitalization of the mouth anatomy. At step 1206, the 3D model of the intraoral scanner is used to design a frame and tooth segments for the RPD. At step 1208, a frame and tooth segment(s) and clasp(s) are 3D printed using the 3D model. At step 1210, the tooth segments and clasps are assembled to the frame.

The clasp has a tail section that has a specific design that allows it only to be adhered and related in one direction to the frame of the RPD. By selecting a virtual clasp corresponding to a selected pre-made physical clasp and using the virtual clasp in the design of the 3D digital model of the RPD to be fabricated, this eliminates the need for the stone model. Relating a clasp into an acrylic base of an RPD without a stone model is an advantage of the method of fabricating an RPD of the present disclosure. The tail section of the clasp is bent in a specific geometry that matches the cut out somewhere in the acrylic base of the RPD that allows it only to go in one direction. The clasp may be embedded in the base, the tooth segment, the frame itself, or any combination thereof.

The resulting RPD meets all the criteria for the ideal digital removable prosthetic. It can be made from an intraoral scan alone. It can be made in a small number of appointments. It requires no shipping by the dentist at all. It can be 3D printed (e.g., by fused deposition modeling, stereolithography, or selective laser sintering) or milled. It can be made from several materials. It can include a rigid or flexible major connector from the same design. It requires no stone model for assembly. Most importantly, the new design results in the most marketable RPD that can be priced to match current models for the dentist.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment,” or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

We claim:

1. A method of fabricating a removable dental appliance that engages an abutment tooth, the method comprising:
(a) acquiring 3D digital image data of a patient's mouth and dentition;
(b) creating a 3D digital model of a removable dental appliance to be fabricated wherein the 3D digital model incorporates a model clasp selected from a digital library of virtual clasps, each of the virtual clasps corresponding to an associated physical clasp of a plurality of physical clasps structured to accommodate functional movement of the removable dental appliance to be fabricated with respect to the abutment tooth when the removable dental appliance to be fabricated is placed in the patient's mouth;
(c) fabricating a portion of the removable dental appliance from the 3D digital model, the portion of the removable dental appliance including an attachment section;
(d) selecting one physical clasp of the plurality of physical clasps that corresponds to the model clasp; and
(e) inserting a tail section of a selected physical clasp into mounting structure in the attachment section of the removable dental appliance, the tail section being dimensioned to matingly engage the mounting structure in the attachment section to assemble the selected physical clasp to the attachment section of the removable dental appliance.

2. The method of claim 1 wherein:
each of the plurality of physical clasps comprises at least one section comprising wrought wire.

3. The method of claim 2 wherein:
each of the plurality of physical clasps comprises a ball clasp.

4. The method of claim 3 wherein:
each ball clasp comprises a horizontal member attached to a tail component and a vertical member that terminates in a ball.

5. The method of claim 4 wherein:
the horizontal member of at least two of the plurality of physical clasps has a different length.

6. The method of claim 4 wherein:
the vertical member of at least two of the plurality of physical clasps has a different length.

7. The method of claim 4 wherein:
the tail component of at least one of the plurality of physical clasps has a looped configuration or a bent configuration.

8. The method of claim 4 wherein:
the tail component of at least one of the plurality of physical clasps is stamped.

9. The method of claim 8 wherein:
the tail component of at least one of the plurality of physical clasps includes one or more cutout regions within a perimeter of the tail component.

10. The method of claim 4 wherein:
the tail component of at least one of the plurality of physical clasps has an abraded surface.

11. The method of claim 4 wherein:
the tail component of at least one of the plurality of physical clasps has a serrated edge.

12. The method of claim 2 wherein:
each of the plurality of physical clasps comprises a C-clasp.

13. The method of claim 12 wherein:
each C-clasp comprises a ridgelap segment attached to a tail component and a curved portion.

14. The method of claim 13 wherein:
the curved portion at least one of the plurality of physical clasps terminates in a ball.

15. The method of claim 13 wherein:
the ridgelap segment of at least two of the plurality of physical clasps has a different length.

16. The method of claim 13 wherein:
the curved portion of at least two of the plurality of physical clasps has a different length.

17. The method of claim 13 wherein:
the tail component of at least one of the plurality of physical clasps has a looped configuration or a bent configuration.

18. The method of claim 13 wherein:
the tail component of at least one of the plurality of physical clasps is stamped.

19. The method of claim 13 wherein:
the tail component of at least one of the plurality of physical clasps includes one or more cutout regions within a perimeter of the tail component.

20. The method of claim 13 wherein:
the tail component of at least one of the plurality of physical clasps has an abraded surface.

21. The method of claim 13 wherein:
the tail component of at least one of the plurality of physical clasps has a serrated edge.

22. The method of claim 1 wherein:
each of the plurality of physical clasps comprises a reciprocal arm and a retentive arm wherein at least one of the reciprocal arm and the retentive arm comprises wrought wire.

23. The method of claim 1 wherein:
step (e) comprises curing a resin in the mounting structure in the attachment section to assemble the selected physical clasp to the attachment section of the removable dental appliance.

24. The method of claim 1 wherein:
step (c) comprises fabricating the portion of the removable dental appliance using 3D printing.

25. The method of claim 1 wherein:
step (c) comprises fabricating the portion of the removable dental appliance using a milling procedure.

26. The method of claim 25 wherein:
step (e) comprises inserting the tail section of the selected physical clasp into mounting structure in the attachment section of the removable dental appliance during the milling procedure.

27. The method of claim 1 wherein:
each of the plurality of physical clasps is selected from the group consisting of ball clasps, C-clasps, wing clasps, I-bar clasps, Y-bar clasps, combination clasps, RPI clasps, RPA clasps, rest seats, orthodontic clasps, Adams clasps, Hawley retainers, and palatal expanders.

28. The method of claim 1 wherein:
the removable dental appliance is a removable partial denture comprising (i) a frame sized and shaped to conform to a mouth inner surface; and (ii) a tooth segment assembled to the frame, and
the attachment section is a base section of the frame.

29. The method of claim 28 wherein:
the mounting structure in the attachment section comprises a cut out.

30. The method of claim 1 wherein:
the 3D digital model incorporates a model seat selected from a digital library of virtual seats, each of the virtual seats corresponding to an associated physical seat of a plurality of physical seats.

31. The method of claim 30 further comprising:
(f) selecting one physical seat of the plurality of physical seats that corresponds to the model seat; and
(g) inserting a tail section of a selected physical seat into mounting structure in the attachment section of the removable dental appliance, the tail section of the selected physical seat being dimensioned to matingly engage the mounting structure in the attachment section to assemble the selected physical seat to the attachment section of the removable dental appliance.

32. The method of claim 1 wherein:
step (c) comprises fabricating the portion of the removable dental appliance using 3D color printing.

33. The method of claim 1 wherein:
the removable dental appliance is a removable partial denture comprising (i) a frame sized and shaped to conform to a mouth inner surface; and (ii) a tooth segment assembled to the frame,
the attachment section is a base section of the frame, and
step (e) comprises inserting the tail section of the selected physical clasp into mounting structure in a buccal flange of the base section of the frame.

34. The method of claim 1 wherein:
the removable dental appliance is a removable partial denture comprising (i) a frame sized and shaped to conform to a mouth inner surface; and (ii) a tooth segment assembled to the frame,
the attachment section is a base section of the frame, and
step (e) comprises inserting the tail section of the selected physical clasp into mounting structure in a lingual flange of the base section of the frame.

35. A method of fabricating a removable dental appliance that engages an abutment tooth, the method comprising:
(a) acquiring 3D digital image data of a patient's mouth and dentition;
(b) creating a 3D digital model of a removable dental appliance to be fabricated wherein the 3D digital model incorporates a model seat selected from a digital library of virtual seats, each of the virtual seats corresponding to an associated physical seat of a plurality of physical seats;
(c) fabricating a portion of the removable dental appliance from the 3D digital model, the portion of the removable dental appliance including an attachment section;
(d) selecting one physical seat of the plurality of physical seats that corresponds to the model seat; and
(e) inserting a tail section of a selected physical seat into mounting structure in the attachment section of the removable dental appliance, the tail section being dimensioned to matingly engage the mounting structure in the attachment section to assemble the selected physical seat to the attachment section of the removable dental appliance.

36. The method of claim 35 further comprising:
(f) creating a preparation in the abutment tooth wherein the preparation has an interior surface for matingly engaging an outer surface of the selected physical seat.

37. The method of claim 36 wherein:
step (f) comprises creating the preparation in the abutment tooth using a dental bur.

38. The method of claim 37 wherein:
the dental bur has a cutting edge geometry that matches the outer surface of the selected physical seat.

39. The method of claim 37 wherein:
the dental bur has a depth stop.

40. The method of claim 35 further comprising:
(f) providing a plurality of dental burs, one or more of the dental burs having a cutting edge geometry that matches an outer surface of one or more of the plurality of physical seats; and
(g) creating a preparation in the abutment tooth using a least one of the dental burs, wherein the preparation has an interior surface for matingly engaging an outer surface of the selected physical seat.

41. The method of claim 35 wherein:
the tail section of at least one of the plurality of physical seats has a looped configuration or a bent configuration.

42. The method of claim 35 wherein:
the tail section of at least one of the plurality of physical seats is stamped.

43. The method of claim 35 wherein:
the tail section of at least one of the plurality of physical seats includes one or more cutout regions within a perimeter of the tail component.

44. The method of claim 35 wherein:
the tail section of at least one of the plurality of physical seats has an abraded surface.

45. The method of claim 35 wherein:
the tail section of at least one of the plurality of physical seats has a serrated edge.

46. The method of claim 35 wherein:
step (e) comprises curing a resin in the mounting structure in the attachment section to assemble the selected physical seat to the attachment section of the removable dental appliance.

47. The method of claim 35 wherein:
step (c) comprises fabricating the portion of the removable dental appliance using 3D printing.

48. The method of claim 35 wherein:
step (c) comprises fabricating the portion of the removable dental appliance using a milling procedure.

49. The method of claim 35 wherein:
step (e) comprises inserting the tail section of the selected physical seat into mounting structure in the attachment section of the removable dental appliance during the milling procedure.

50. The method of claim 49 wherein:
the mounting structure in the attachment section comprises a cut out.

51. The method of claim 35 wherein:
the removable dental appliance is a removable partial denture comprising (i) a frame sized and shaped to conform to a mouth inner surface; and (ii) a tooth segment assembled to the frame, and
the attachment section is a base section of the frame.

52. The method of claim 35 wherein:
the 3D digital model incorporates a model clasp selected from a digital library of virtual clasps, each of the virtual clasps corresponding to an associated physical clasp of a plurality of physical clasps.

53. The method of claim 52 further comprising:
(f) selecting one physical clasp of the plurality of physical clasps that corresponds to the model clasp; and
(g) inserting a tail section of a selected physical clasp into mounting structure in the attachment section of the removable dental appliance, the tail section of the selected physical clasp being dimensioned to matingly engage the mounting structure in the attachment section to assemble the selected physical clasp to the attachment section of the removable dental appliance.

54. The method of claim 35 wherein:
step (c) comprises fabricating the portion of the removable dental appliance using 3D color printing.

55. A method of fabricating a removable partial denture that engages an abutment tooth, the method comprising:
(a) acquiring 3D digital image data of a patient's mouth and dentition;
(b) creating a 3D digital model of a removable partial denture to be fabricated wherein the 3D digital model incorporates a model clasp structured to accommodate functional movement of the removable partial denture to be fabricated with respect to the abutment tooth when the removable partial denture to be fabricated is placed in the patient's mouth;
(c) fabricating a portion of the removable partial denture from the 3D digital model, the portion of the removable partial denture including a tooth attachment section and at least one artificial tooth;
(d) fabricating an additional artificial tooth having an attached physical clasp that corresponds to the model clasp; and
(e) assembling the additional artificial tooth in the tooth attachment section of the removable partial denture.

56. The method of claim 55 wherein:
the physical clasp comprises at least one section comprising wrought wire.

57. The method of claim 55 wherein:
the physical clasp comprises a ball clasp.

58. The method of claim 55 wherein:
the physical clasp comprises a C-clasp.

59. The method of claim 55 wherein:
the physical clasp comprises a reciprocal arm and a retentive arm wherein at least one of the reciprocal arm and the retentive arm comprises wrought wire.

60. The method of claim 55 wherein:
step (e) comprises curing a resin in the tooth attachment section to assemble the physical clasp in the tooth attachment section of the removable partial denture.

61. The method of claim 55 wherein:
step (c) comprises fabricating the portion of the removable partial denture using 3D printing.

62. The method of claim 55 wherein:
step (c) comprises fabricating the portion of the removable partial denture using color 3D printing.

63. The method of claim 55 wherein:
step (c) comprises fabricating the portion of the removable partial denture using milling.

64. The method of claim 55 wherein:
step (d) comprises fabricating the additional artificial tooth having the attached physical clasp using 3D printing.

65. The method of claim 55 wherein:
step (d) comprises fabricating the additional artificial tooth having the attached physical clasp using color 3D printing.

66. The method of claim 55 wherein:
step (d) comprises fabricating the additional artificial tooth having the attached physical clasp using milling.
